(19) United States
(12) Patent Application Publication
(10) Pub. No.: US 2007/0173686 A1
(43) Pub. Date: Jul. 26, 2007

(54) Capsular endoscope device with an orientation/release mechanism

(76) Inventors: Chun Min Lin, Hsinchu City (TW); Shih Chieh Lu, Hsinchu City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/434,772

(22) Filed: May 17, 2006

(30) Foreign Application Priority Data
Jan. 24, 2006 (TW) 095102695

(51) Int. Cl. A61B 1/00

(52) U.S. Cl. 600/102; 600/114

(57) ABSTRACT

The present invention discloses a capsular endoscope device with an orientation/release mechanism. The capsular endoscope device of the present invention has a casing with the front end thereof having a hose, and the front end of the hose has a capsular-endoscope control device, which a capsular endoscope can be attached to. A deflection control device, which is installed to the casing, is used to deflect the capsular-endoscope control device so that the capsular endoscope will be moved to have an elevation angle. A rotation/release control device, which is installed inside the casing, is used to rotate the capsular-endoscope control device so that the capsular endoscope will be rotated also. The rotation/release control device can also release the capsular endoscope from the capsular-endoscope control device. Therefore, the capsular endoscope device with an orientation/release mechanism proposed by the present invention can enable the user to control the orientation of the camera lens of the capsular endoscope to fully inspect the tissue inside a human body cavity.

### BACKGROUND OF THE INVENTION

1. Field of the Invention

The present invention relates to a capsular endoscope device, particularly to a capsular endoscope device with an orientation/release mechanism.

2. Description of the Related Art

Endoscope systems are widely used to inspect the digestive tract. In a conventional endoscope system, a hose with a miniature sensor reaches the deep of the digestive tract of a human body to obtain tissue images for identifying the nidus type and the development thereof.

Most of the current endoscope systems adopt electronic endoscopes. As electronic endoscopes are very expensive, they must be used repeatedly. Thus, cross infection may occur among patients when endoscopes are not fully disinfected. Current electronic endoscopes for the digestive tract can only, inspect the oesophagus, the stomach, and the large intestine. As to the small intestine, a current electronic endoscope can only reach the front 90 cm thereof. Thus, the most portion of the small intestine cannot be inspected by a current electronic endoscope.

Therefore, a capsular endoscope system, which can wireless transmit tissue images, was developed to solve the abovementioned problems. However, such a capsular endoscope is disadvantaged in lacking controllability. Thus, the capsular endoscope is hard to fully obtain necessary information. Refer to FIG. 1 and FIG. 2. In order to effectively control the location where the capsular endoscope reaches, two PCT (Patent Cooperation Treaty) patents of Application No. WO9932028 and Application No. WO0189596 respectively proposes a clamp/release mechanism 6 and a hydraulic-release mechanism 8 for a capsular endoscope 2 and a capsular endoscope 4. Although those two technologies can control the location of the capsular endoscope, they cannot control the orientation of the camera lens of the capsular endoscope yet. Therefore, when the capsular endoscope is placed in a larger cavity of a human body, such as the stomach, it is hard to fully inspect the larger cavity, and some portions thereof may be neglected, and the usage of the capsular endoscope is thus limited.

Accordingly, the present invention proposes a capsular endoscope device with an orientation/release mechanism to solve the abovementioned problems.

### SUMMARY OF THE INVENTION

The primary objective of the present invention is to provide a capsular endoscope device with an orientation/release mechanism, wherein the user can control the orientation of the capsular endoscope when the capsular endoscope has been placed in a cavity of a human body, and the user can further release the capsular endoscope to perform the succeeding inspection.

Another objective of the present invention is to provide a capsular endoscope device, wherein the user can control the orientation of the capsular endoscope to fully observe the tissue of a human cavity so that more useful information of the human cavity can be obtained.

Still another objective of the present invention is to provide a capsular endoscope device, wherein the inspections of the larger organ and the small intestine can be completed with one step of the digestive-tract inspection.

Further another objective of the present invention is to provide a capsular endoscope device with an orientation/release mechanism, which is advantaged in structural simplification, low cost, and disposability.

To achieve the abovementioned objectives, the present invention proposes a capsular endoscope device with an orientation/release mechanism. The capsular endoscope device of the present invention has a casing with the front end thereof having a hose, and the front end of the hose has a capsular-endoscope control device, which a capsular endoscope can be attached to. A deflection control device installed to the casing and a rotation/release control device installed inside the casing are used to control the capsular-endoscope control device. The deflection control device further comprises: a winding wheel element, installed inside the casing; a connecting line, coupled to the winding wheel element, and passing through the hose to connect with the capsular-endoscope control device; and an operation element, coupled to the winding wheel element, and used to control the winding wheel element. The deflection control device controls the deflection of the capsular-endoscope control device via releasing or rewinding the connecting line, and thus, the capsular endoscope is also moved. The rotation/release control device further comprises: a connecting element, passing through the hose to connect with the capsular-endoscope control device; and an operation element, inserted into the casing, coupled to the connecting element, and able to move back and forth. When the operation element of the rotation/release control device is rotated, the connecting element is actuated to rotate, and the connecting element further drives the capsular endoscope to rotate also. When the operation element of the rotation/release control device is pressed into the casing, the connecting element is actuated to push the capsular endoscope out of the capsular-endoscope control device.

To enable the objectives, technical contents, characteristics, and accomplishments of the present invention to be more easily understood, the embodiments of the present invention are to be described in detail in cooperation with the attached drawings below.

### BRIEF DESCRIPTION OF THE DRAWINGS

FIG. 1 is a diagram showing a conventional endoscope with a clamp/release mechanism.

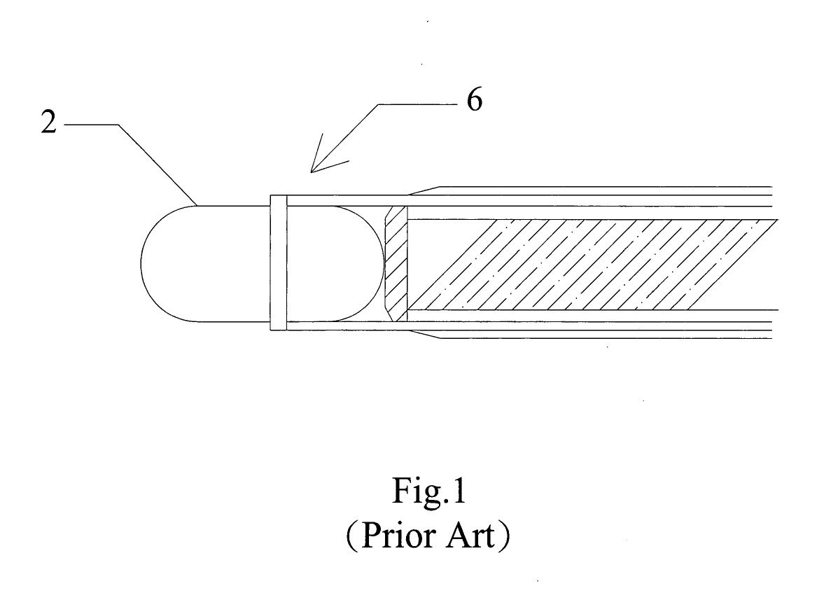

FIG. 2 is a diagram showing a conventional endoscope with a hydraulic-release mechanism.

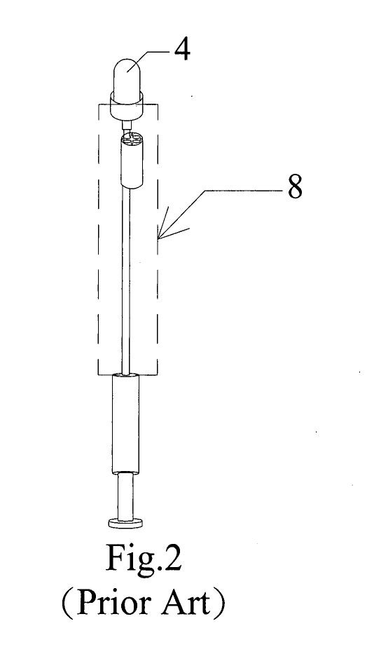

FIG. 3 is a diagram schematically showing the appearance of the present invention.

FIG. 4 is a diagram schematically showing an exploded view of the present invention.

FIG. 5(a) is a diagram schematically showing a partially-enlarged view of the capsular-endoscope control device according to the present invention.

FIG. 5(b) is a diagram schematically showing a partially-enlarged view of the casing according to the present invention.

FIG. 6 is a diagram schematically showing the deflection of the capsular-endoscope control device according to the present invention.

FIG. 7 is a diagram schematically showing the rotation of the capsular-endoscope control device according to the present invention.

FIG. 8 is a diagram schematically showing that the rotation/release control device pushes the capsular endoscope out of the capsular-endoscope control device according to the present invention.

### DETAILED DESCRIPTION OF THE INVENTION

The present invention provides an orientation/release mechanism for a capsular endoscope device. When the capsular endoscope has been placed in a cavity of a human body, the user can control orientation the capsular endoscope to inspect the larger organ in the human cavity, and then, the capsular endoscope is released from the orientation/release mechanism to perform the succeeding small-intestine inspection.

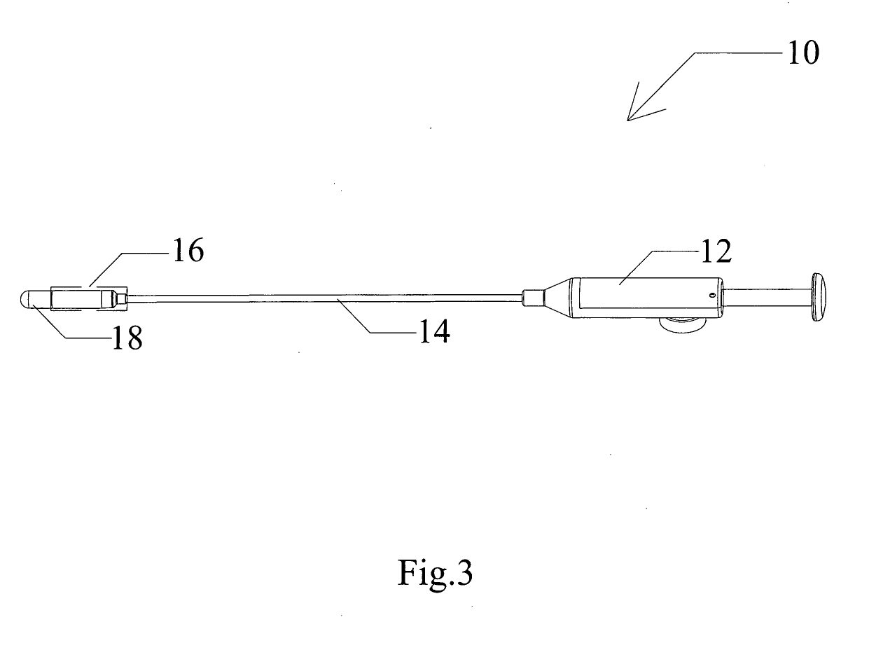

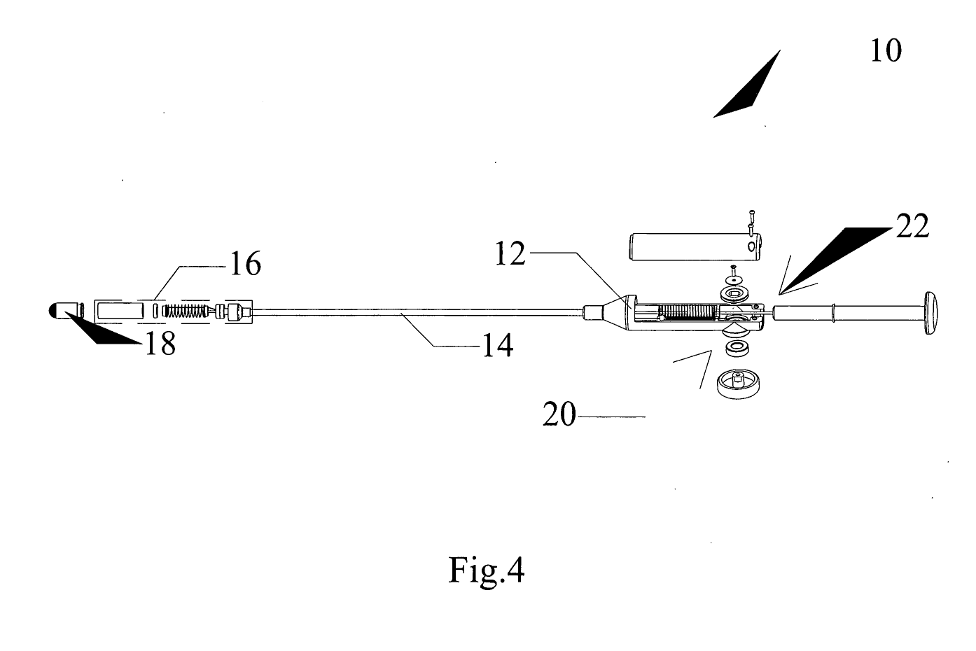

Refer to FIG. 3 and FIG. 4 diagrams respectively schematically showing the appearance and an exploded view of the present invention. The capsular endoscope device 10 of the present invention comprises a casing 12. The front end of the casing 12 has a hose 14, and the hose 14 may be a single-channel hose or a multi-channel hose. The front end of the hose 14 has a capsular-endoscope control device 16, and a capsular endoscope 18 can be attached to the capsular-endoscope control device 16. The capsular endoscope device 10 also comprises: a deflection control device 20 and a rotation/release control device 22. The deflection control device 20 is installed to casing 12 and can control the capsular-endoscope control device 16 to perform deflection actions. The rotation/release control device 22 is installed inside the casing 12 and can control the capsular-endoscope control device 16 to perform rotation actions. Besides, conductive cables, such as power cables and signal cables, pass through the casing 12 and the hose 14 to connect with the capsular endoscope 18, and the disengagement of the connection between the cables and the capsular endoscope 18 can be controlled externally.

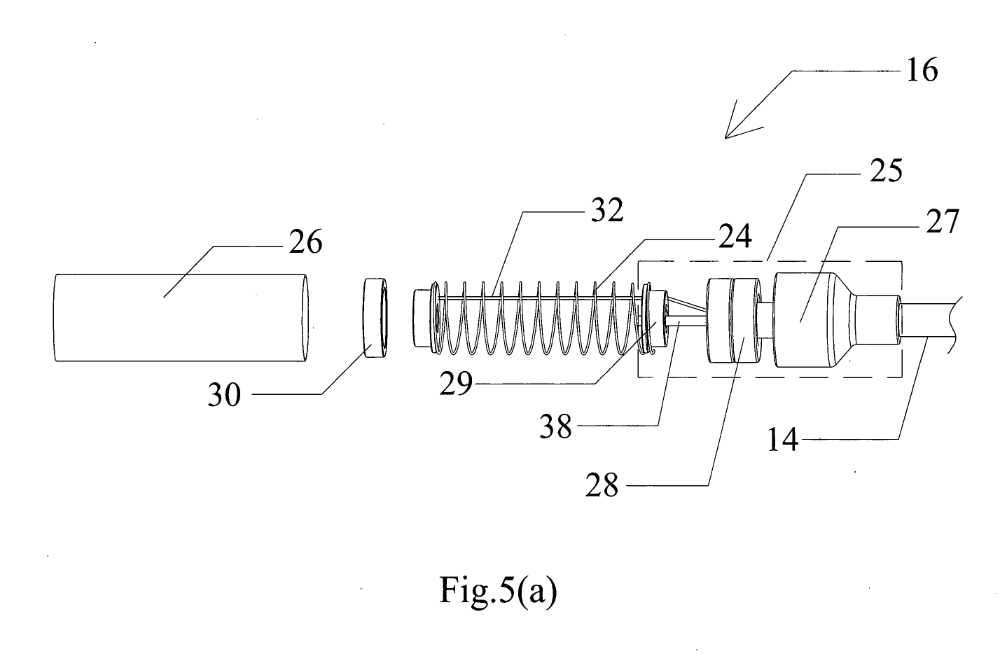

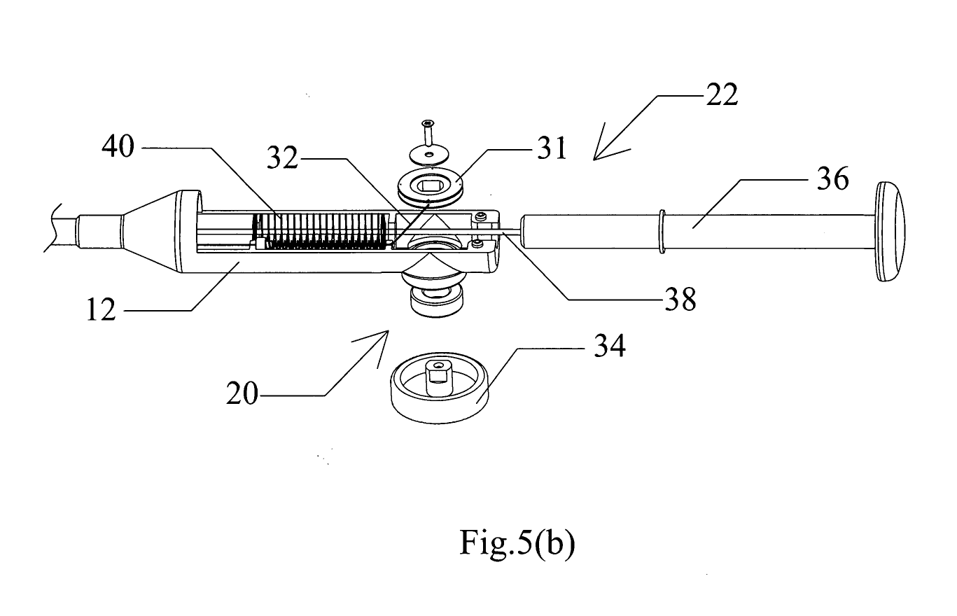

Refer to FIG. 5(a) and FIG. 5(b) diagrams respectively schematically showing partially-enlarged views of the capsular-endoscope control device and the casing. As shown in FIG. 5(a), in the capsular-endoscope control device 16, a first elastic element 24, such as a spring, is fixedly installed to a rotation member 25 and sleeved inside a flexible casing 26. The rotation member 25 further comprises: a fixing element 27, used to fix the hose 14; an installation element 28, disposed at the front end of the fixing element 27, and used to install the flexible casing 26; and an actuating element 29, installed inside the installation element 28, and coupled to the first elastic element 24. A capsular endoscope can be attached to the flexible casing 26 of the capsular-endoscope control device 16, and the first elastic element 24 can be used to actuate the flexible casing 26. In order to actuate the flexible casing 26 more easily, the present invention further installs a drive element 30 at the front end of the first elastic element 24, and the size of the drive element 30 is slightly smaller that the inner diameter of the flexible casing 26.

As shown in FIG. 5(a) and FIG. 5(b), the deflection control device 20 of the present invention further comprises: a winding wheel element 31, installed inside the casing 12; an operation element 34, coupling to the winding wheel element 31, and used to control the winding wheel element 31, and a connecting line 32, such as a metallic thread, with one end of the connecting line 32 coupled to the winding wheel element 31 and another end passing through the hose 14 to connect to the front end of the first elastic element 24 of the capsular-endoscope control device 16. The rotation/release control device 22 of the present invention further comprises: an operation element 36, inserted into the casing 12, and able to move back and forth; and a connecting element 38, with one end thereof coupled to the operation element 36 and another end passing through the hose 14 to connect with the actuating element 29 of the rotation member 25. The connecting element 38 is made of a metallic material, such as iron; the connecting element 38 has enough rigidity to transmit the driving torque to the actuating element 29 and has enough flexibility to adapt to the flexible hose 14. In order to restore the operation element 36 to its original position when it has been pressed into the casing 12, a second elastic element 40, such as a spring, is installed inside the casing 12. The second elastic element 40 is pressed against the operation element 36. When the operation element 36 has been pressed into the casing 12, the elastic force of the second elastic element 40 will restore the operation element 36 to its original position.

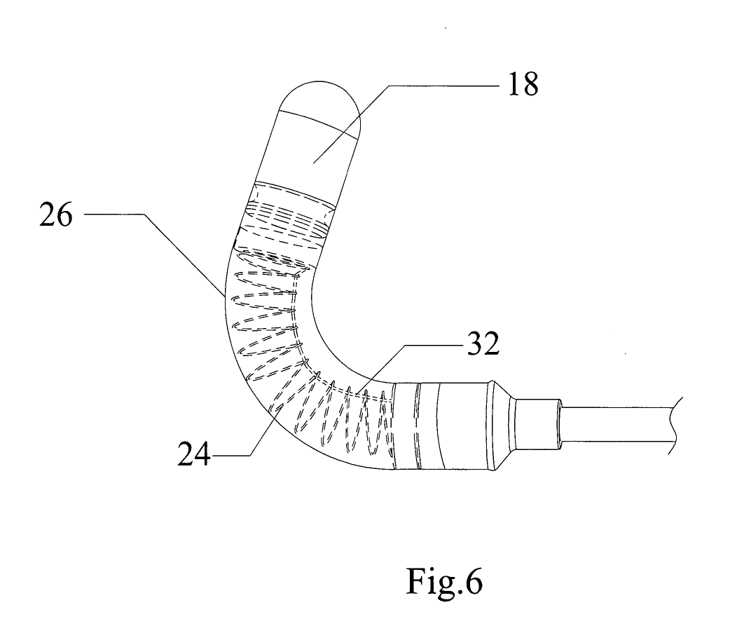

Refer to FIG. 4, FIG. 5(a) and FIG. 5(b) again for the operation method of the present invention. When the capsular endoscope has been placed in a cavity of a human body, the user can utilizes the deflection control device 20 and the rotation/release control device 22 to operate the capsular-endoscope control device 16. Refer to FIG. 6 also for the operation of the deflection control device 20. When the operation element 34 drives the winding wheel element 31 to rotate, the connecting line 32 will be rewound or released. When the connecting line 32 is rewound by the winding wheel element 31, the pulling force of the connecting line 32 will deflect the first elastic element 24, and thus, the flexible casing 26 is also deflected. When the connecting line 32 is released, the flexible casing 26 is restored to its original shape by the elastic force of the first elastic element 24 and the connecting line 32. Thereby, the capsular-endoscope control device 16 can be deflected via operating the operation element 34, and thus, the capsular endoscope 18 is also moved to have an elevation angle.

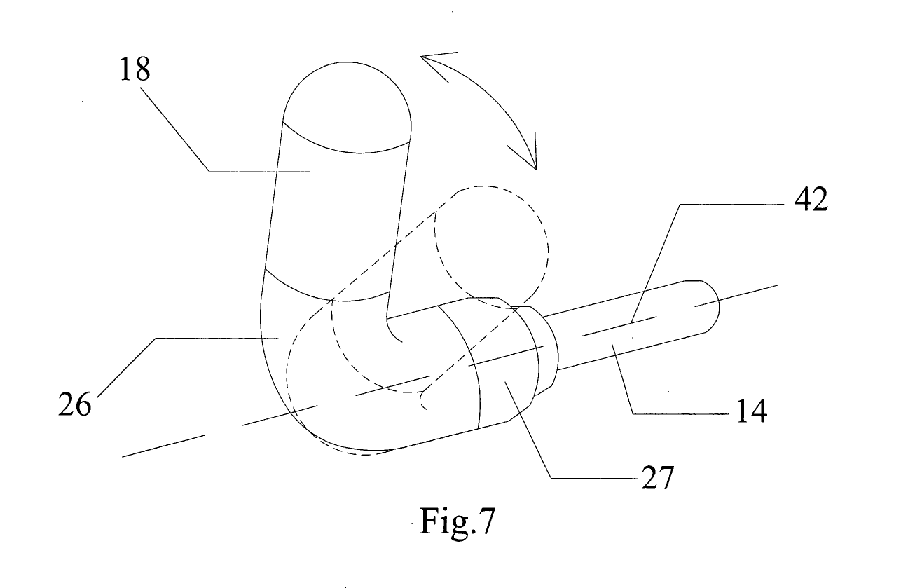

Refer to FIG. 7. When the operation element 36 is rotated, the connecting element 38 is also driven to rotate, and the connecting element 38 further drives the actuating element 29 of the rotation member 25 to rotate. Thus, the first elastic element 24, which is coupled to the actuating element 29, also drives the deflected flexible casing 26 to rotate around the central axis 42 of the rotation member 25, and the capsular endoscope 18, which has been attached to the deflected flexible casing 26, also rotates around the central axis 42. Therefore, the user can utilize the deflection control device 20 and the rotation/release control device 22 to arbitrarily control the orientation and the elevation angle of the capsular endoscope 18 so that the tissue of a human cavity can be fully inspected and more information can also be obtained with the capsular endoscope 18 of the present invention.

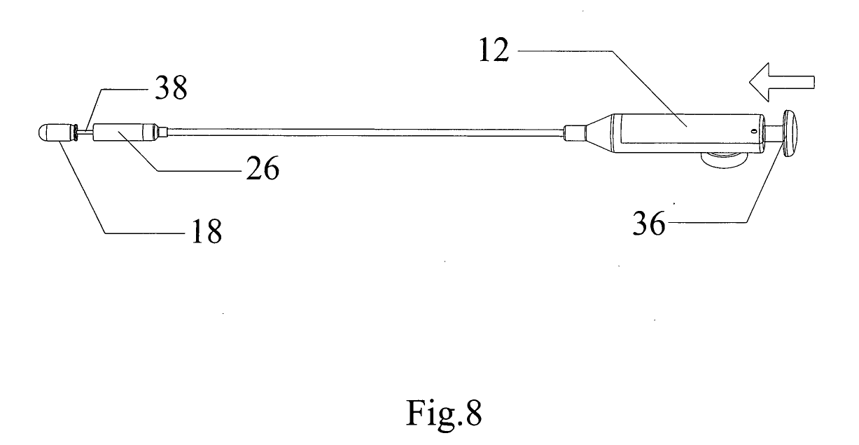

Refer to FIG. 8. When the operation element 36 is pressed into the casing 12, the connecting element 38 is also pressed forward to push the bottom of the capsular endoscope 18, and thus, the capsular endoscope 18 is pushed out of the flexible casing 26 and released from the control of the capsular-endoscope control device 16. Then, the user withdraws the capsular-endoscope control device 16 and the hose 14 from the human cavity. Thus, after the larger organ has been inspected, the user can still use the capsular endoscope 18 to wirelessly inspect the small intestine. Therefore, the present invention is advantaged in that the inspections of the larger organ and the small intestine can be completed with one step of the digestive-tract inspection. Further the present invention is also advantaged in structural simplification, low cost, and disposability.

In the present invention, the user can utilize the deflection control device and the rotation/release control device to control the movement of the capsular endoscope. Therefore, the capsular endoscope device with an orientation/release mechanism of the present invention not only can overcome the problem that the conventional technologies cannot control the orientation of the camera lens of a capsular endoscope, but also can fully inspect the tissue of a human cavity and obtain more useful information.

Those embodiments described above are to clarify the present invention in order to enable the persons skilled in the art to understand, make and use the present invention; however, it is not intended to limit the scope of the present invention; any equivalent modification and variation according to the spirit of the present invention is to be also included within the scope of the claims stated below.

What is claimed is:

1. A capsular endoscope device with an orientation/release mechanism, comprises:
a casing, having a hose at the front end thereof;
a capsular-endoscope control device, disposed at the front end of said hose, and able to install a capsular endoscope;
a deflection control device, installed to said casing, used to deflect said capsular-endoscope control device so that said capsular endoscope will be driven to move by said capsular-endoscope control device; and
a rotation/release control device, installed inside said casing, used to drive said capsular-endoscope control device to rotate so that said capsular endoscope will be driven to rotate by said capsular-endoscope control device, and used to release said capsular endoscope from said capsular-endoscope control device.

2. The capsular endoscope device according to claim 1, wherein said hose may be a single-channel hose or a multi-channel hose.

3. The capsular endoscope device according to claim 1, wherein said capsular-endoscope control device further comprises: a rotation member; a first elastic element, fixedly installed to said rotation member; and a flexible casing, sleeving said first elastic element.

4. The capsular endoscope device according to claim 3, wherein said first elastic element is a spring.

5. The capsular endoscope device according to claim 3, wherein said rotation member further comprises:
a fixing element, used to fix said hose;
an installation element, disposed at the front end of said fixing element, and used to install said flexible casing; and
an actuating element, installed inside said installation element, and coupled to said first elastic element.

6. The capsular endoscope device according to claim 3, wherein a drive element is further installed at the front end of said first elastic element, and the size of said drive element is slightly smaller that the inner diameter of said flexible casing so that said first elastic element can drive said flexible casing more easily.

7. The capsular endoscope device according to claim 1, wherein deflection control device further comprises:
a winding wheel element, installed inside said casing;
a connecting line, installed to said winding wheel element, and passing through said hose to connect with said capsular-endoscope control device;
an operation element, used to control the rotation of said winding wheel element, and able to rewind or release said connecting line so that said capsular-endoscope control device will be deflected and said capsular endoscope will be moved.

8. The capsular endoscope device according to claim 7, wherein said connecting line is a metallic thread.

9. The capsular endoscope device according to claim 1, wherein said rotation/release control device further comprises:
a connecting element, passing through said hose to connect with said capsular-endoscope control device; and
an operation element, inserted into said casing, able to move back and forth, and coupled to said connecting element so that when said operation element is rotated, said operation element can actuate said connecting element to rotate, and said connecting element further drive said capsular-endoscope control device and said capsular endoscope to rotate; when said operation element is pressed into said casing, said connecting element can push said capsular endoscope out of said capsular-endoscope control device.

10. The capsular endoscope device according to claim 9, wherein said connecting element is made of a metallic material.

11. The capsular endoscope device according to claim 10, wherein said metallic material is iron.

12. The capsular endoscope device according to claim 9, wherein said casing further comprises a second elastic element, and said second elastic element is pressed against said operation element; when said operation element has been pressed into said casing, the elastic force of said second elastic element will restore said operation element to its original position.

13. The capsular endoscope device according to claim 12, wherein said second elastic element is a spring.
